(19) United States
(12) Patent Application Publication
(10) Pub. No.: US 2008/0058841 A1
(43) Pub. Date: Mar. 6, 2008

(54) SYSTEM AND METHOD FOR MARKING CORNEAL TISSUE IN A TRANSPLANT PROCEDURE

(76) Inventors: Ronald M. Kurtz, Irvine, CA (US); Melvin A. Sarayba, Ladera Ranch, CA (US); Roger Steinert, Laguna Beach, CA (US)

Correspondence Address: Towsend and Towsend and Crew LLP/IntraLase Corp., Two Embarcadero Center, Eighth Floor, San Francisco, CA, 94111, US

(21) Appl. No.: 11/469,901

(22) Filed: Sep. 5, 2006

(51) Int. Cl. A61F 9/01

(52) U.S. Cl. 606/166

(57) ABSTRACT

A system and method for marking corneal tissue in a transplant procedure is disclosed. Sidecut incisions are made in each of the donor cornea and the recipient cornea. Crosscut incisions are made across the sidecut incision in each cornea. Corneal tissue which is at least partially bounded by the sidecut incision is resected from the recipient cornea. Part of the crosscut incision made in the recipient cornea extends beyond the resected corneal tissue. Donor tissue which is at least partially bounded by the sidecut incision is resected from the donor cornea. Part of the crosscut incision made in the donor cornea is in the resected donor tissue. The donor tissue is then grafted into the recipient cornea.

### BACKGROUND OF THE INVENTION

1. Field of the Invention

The field of the present invention is systems and procedures for transplanting corneas.

2. Background

A variety of techniques presently exist for performing both full thickness corneal transplants and lamellar corneal transplants. The tools used for the different techniques range from the traditional trephine to the more advanced laser surgical systems. Regardless of the technique or tool used for either type of transplant procedure, the overarching goal remains to provide the recipient with new corneal tissue for the best possible post-operative optical quality. One of the most common post-operative complications is induced astigmatism, and improvements on current techniques are needed to minimize, if not eliminate, this common problem.

### SUMMARY OF THE INVENTION

The present invention is directed toward a system and method for marking corneal tissue in a transplant procedure. In the system, a surgical laser emits a pulsed laser beam which is directed into the cornea by a focusing assembly. An interface provides a plurality of incision patterns for selection of a sidecut pattern and an a quantity of crosscut incisions. Alternatively, the interface may provide a plurality of incision patterns, at least one of which is pre-configured with crosscuts. The selected sidecut pattern and the quantity of crosscut incisions are received by a controller which employs the focusing assembly to move the focal point of the pulsed laser beam and incise corneal tissue according to the sidecut pattern. The controller uses the quantity of crosscut incisions to determine where to place crosscut incisions across the sidecut incision. Preferably, the crosscut incisions are equally spaced about the sidecut incision.

In the method, a sidecut incision is made in each of the donor cornea and the recipient cornea. One or more crosscut incisions are made across the sidecut incision in each cornea. Corneal tissue, which is at least partially bounded by the sidecut incision made in the recipient cornea, is resected from the recipient cornea. The crosscut incision made in the recipient cornea extends beyond the resected corneal tissue. Similarly, donor tissue, which is at least partially bounded by the sidecut incision made in the donor cornea, is resected from the donor cornea. The donor tissue includes part of the crosscut incision that was made in the donor cornea. After both pieces of tissue have been resected, the donor tissue is grafted into the donor cornea. Preferably, all of the incisions are made using a surgical laser.

Other optional steps may be added to the above process, either singly or in combination. In a first optional step, the portion of the crosscut incision in the donor tissue is aligned with the portion of the crosscut incision in the recipient cornea during the grafting step. A second optional step which builds upon the first involves placing a suture along the aligned crosscut incisions. In a third optional step, multiple crosscut incisions may be made in each cornea. In a fourth optional step, the combination of incisions in each cornea form an incision pattern, with the incision pattern in the recipient cornea matching the incision pattern in the donor cornea. In a fifth optional step, the crosscut incision is made at the anterior corneal surface.

Accordingly, an improved system and method for marking corneal tissue in a transplant procedure is disclosed. Advantages of the improvements will appear from the drawings and the description of the preferred embodiment.

### BRIEF DESCRIPTION OF THE DRAWINGS

In the drawings, wherein like reference numerals refer to similar components:

FIG. 1 is a top plan view of a cornea and the incisions made therein;

FIG. 2 is a sectional view of the cornea of FIG. 1 along the line 2-2;

FIG. 3 is a top plan view of donor tissue grafted into a recipient cornea; and

FIG. 4 is a schematic view of a system for resecting corneal tissue.

### DETAILED DESCRIPTION OF THE PREFERRED EMBODIMENTS

Turning in detail to the drawings, FIG. 1 illustrates the incisions made in a cornea 11 as part of a full thickness corneal transplant procedure. The same incisions are made in both the recipient cornea and the donor cornea. The sidecut incision 13 is made through the full thickness of the cornea 11. However, if a lamellar corneal transplant is performed, then the sidecut is made to the depth of the resection incision made within the stroma. In this instance, where the sidecut incision 13 is a full thickness incision, it enables resection of the corneal tissue 15 from the cornea 11. While the sidecut incision shown is a simple incision running straight from the anterior corneal surface to the posterior corneal surface, more complex sidecut incisions may also be used. Four crosscut incisions 17 are shown, and although any number of crosscut incisions may be used, at least two are preferred, with four or more being more preferred. Preferably, multiple crosscut incisions are equally spaced apart along the sidecut incision.

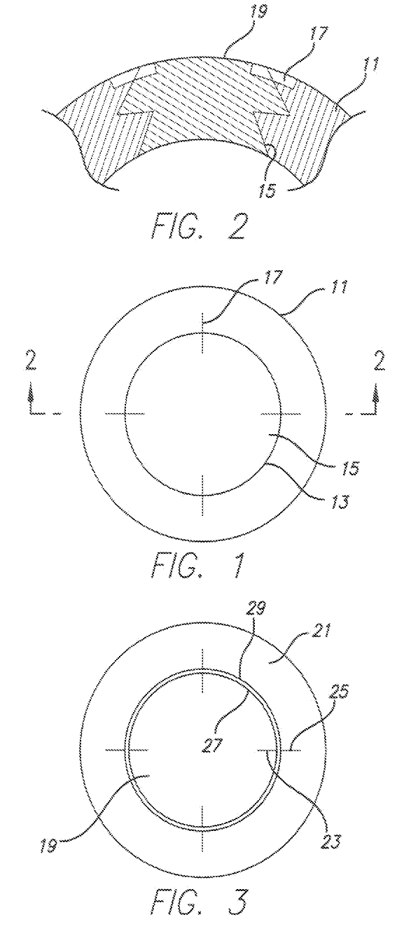

FIG. 2 shows the depth of the crosscuts 17 as compared to the full thickness of the cornea 11. These crosscuts 17 are located at the anterior corneal surface 19 and are deep enough so that the surgeon can easily locate them when performing the graft. The actual depth of the crosscuts 17 may therefore vary depending upon the particular preferences of the attending surgeon. The length of the crosscuts 17 also depends upon the preferences of the attending surgeon. The crosscuts 17 should extend on either side of the sidecut 15 so that the surgeon can easily locate them when performing the graft. Optionally, the crosscuts may be disposed within the stroma, below the epithelium, and need not extend to the anterior corneal surface. This is also left up to the preferences of the attending surgeon.

FIG. 3 shows donor tissue 19 grafted in place within the recipient cornea 21. As indicated above, the positioning of the partial crosscuts 23, 25 within the donor tissue 19 and the recipient tissue 21 facilitates alignment of the two tissues. The amount which these partial crosscuts 23, 25 extend beyond the sidecuts 27, 29 in each of the donor tissue and the recipient cornea, respectively, facilitates placement of sutures. Additionally, a dye or stain, such as those which are well known to skilled artisans, may be used to assist the surgeon in locating and aligning the crosscuts. One suture is preferably placed at the location of each pair of partial crosscuts 23, 25. For a given pair of crosscuts 23, 25, the suture may enter the tissue at any point along the length of each partial sidecut 23, 25. This enables the surgeon to place the sutures as near or as far from the sidecuts 27, 29 as desired.

The combination of all incisions made in each of the recipient cornea and the donor cornea form an incision pattern. It is desirable to have the incision pattern in each of the two corneas be identical and symmetrical. Identical and symmetrical incision patterns enable the donor tissue to be placed in one or more orientations within the recipient cornea and greatly facilitates alignment of the crosscuts in the donor tissue with the crosscuts in the recipient cornea. Moreover, an incision pattern which includes symmetry, especially symmetry in the crosscuts, about multiple axes will help reduce the amount of stress any single suture places on the grafted tissue. Such symmetry most commonly results from the crosscuts being placed at equal intervals about the periphery of the sidecut.

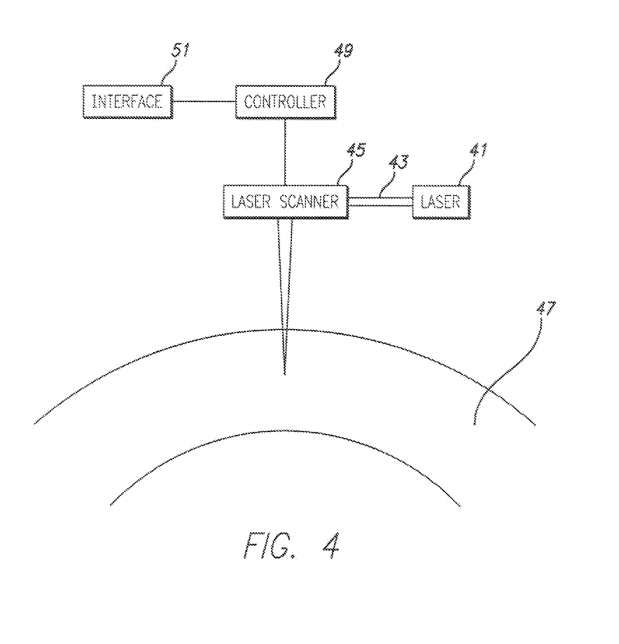

Referring to FIG. 4, a femtosecond surgical laser 41 generates a pulsed laser beam 43 and directs that beam into the focusing assembly 45, which in turn focuses the pulsed beam 43 into the cornea 47. The controller 49 is a programmable computer which precisely controls the location of the beam focal point within the cornea 47 according to parameters received from the surgeon interface 51. The interface 51 presents the surgeon with several incision patterns from which the desired sidecut pattern is selected. In addition, the interface 51 presents the surgeon with options for choosing the number of crosscut incisions. Alternatively, the interface 51 may present the surgeon with resection patterns which are pre-configured with crosscuts. The selected options are sent to the controller, and the controller 49 determines the locations of each crosscut along the sidecut pattern for purposes of controlling the focusing assembly and incising the sidecut pattern, along with the crosscuts, in the cornea.

The surgical laser may be of the type described in U.S. Pat. No. 4,764,930, producing an ultra-short pulsed beam as described in one or both of U.S. Pat. No. 5,984,916 and U.S. Pat. No. RE37,585 to photodisrupt corneal tissues. The focusing assembly may be of the type described in U.S. patent application Ser. No. 11/272,571. The disclosures of the aforementioned patents are incorporated herein by reference in their entirety. Commercial laser systems capable of performing the incisions are available from IntraLase Corp. of Irvine, Calif.

When made with a laser, the incisions will be much less than the thickness of the needles that are typically used to place sutures, but they will still be readily visible to the surgeon (although not necessarily with the naked eye), especially if they are at the anterior corneal surface. Thus, the narrow crosscut incisions made by a laser will generally aid the surgeon in precisely locating the suture in the desired position in both the donor tissue and the recipient cornea.

The surgical laser may be used in conjunction with a contact lens (not shown) which is applied to the anterior corneal surface to deform the cornea. Deformation of the cornea in this manner provides multiple advantages which are well known to skilled artisans. For example, U.S. Pat. No. 5,549,632, which is incorporated herein by reference, describes advantages gained in making laser incisions by deforming the shape of the cornea, particularly by application. U.S. Pat. No. 6,863,667 and U.S. patent application Ser. No. 11/258,399, both of which are incorporated herein by reference, describe patient interface devices which deform the cornea and are used to align the surgical laser with the recipient cornea for purposes of making accurate incisions.

Thus, a system and method for marking corneal tissue in a transplant procedure are disclosed. While embodiments of this invention have been shown and described, it will be apparent to those skilled in the art that many more modifications are possible without departing from the inventive concepts herein. The invention, therefore, is not to be restricted except in the spirit of the following claims.

What is claimed is:

1. A method of transplanting donor tissue from a donor cornea to a recipient cornea, the method comprising:
making a sidecut incision in each of the donor cornea and the recipient cornea;
making a crosscut incision across the sidecut incision in each of the donor cornea and the recipient cornea;
resecting corneal tissue from the recipient cornea, the corneal tissue being at least partially bounded by the sidecut incision in the recipient cornea, wherein a first part of the crosscut incision made in the recipient cornea extends beyond the resected corneal tissue;
resecting donor tissue from the donor cornea, the donor tissue being at least partially bounded by the sidecut incision in the donor cornea, wherein a second part of the crosscut incision made in the donor cornea is in the resected donor tissue; and
grafting the donor tissue into the recipient cornea.

2. The method of claim 1, wherein grafting the donor tissue into the recipient cornea includes aligning the first part of the crosscut incision with the second part of the crosscut incision.

3. The method of claim 1, wherein the crosscut incision made in each cornea does not extend through either cornea.

4. The method of claim 1, wherein making the crosscut incision includes making the crosscut incision at an anterior corneal surface in each of the donor cornea and the recipient cornea.

5. The method of claim 1, wherein the incisions made in the recipient cornea form a first incision pattern, the incisions formed in the donor cornea form a second incision pattern, and the first incision pattern matches the second incision pattern.

6. The method of claim 1 further comprising placing a suture along the aligned parts of the crosscut incisions.

7. The method of claim 1, wherein the incisions are made using a surgical laser.

8. A method of transplanting donor tissue from a donor cornea to a recipient cornea, the method comprising:
making a sidecut incision in each of the donor cornea and the recipient cornea;
making a crosscut incision across the sidecut incision and at an anterior corneal surface in each of the donor cornea and the recipient cornea;
resecting corneal tissue from the recipient cornea, the corneal tissue being at least partially bounded by the sidecut incision in the recipient cornea, wherein a first part of the crosscut incision made in the recipient cornea extends beyond the resected corneal tissue;
resecting donor tissue from the donor cornea, the donor tissue being at least partially bounded by the sidecut incision in the donor cornea, wherein a second part of the crosscut incision made in the donor cornea is in the resected donor tissue; and
grafting the donor tissue into the recipient cornea by aligning the first part of the crosscut incision with the second part of the crosscut incision.

9. The method of claim 8, wherein the crosscut incision made in each cornea does not extend through either cornea.

10. The method of claim 8 further comprising placing a suture along the aligned parts of the crosscut incisions.

11. The method of claim 8, wherein the incisions are made using a surgical laser.

12. A method of transplanting donor tissue from a donor cornea to a recipient cornea, the method comprising:
making a sidecut incision in each of the donor cornea and the recipient cornea;
making a plurality of crosscut incisions across the sidecut incision in each of the donor cornea and the recipient cornea, wherein the incisions made in the recipient cornea form a first incision pattern, the incisions formed in the donor cornea form a second incision pattern, and the first incision pattern matches the second incision pattern;
resecting corneal tissue from the recipient cornea, the corneal tissue being at least partially bounded by the sidecut incision in the recipient cornea, wherein a first part of each crosscut incision made in the recipient cornea extends beyond the resected corneal tissue;
resecting donor tissue from the donor cornea, the donor tissue being at least partially bounded by the sidecut incision in the donor cornea, wherein a second part of each crosscut incision made in the donor cornea is in the resected donor tissue; and
grafting the donor tissue into the recipient cornea by aligning the first part of each crosscut incision in the recipient cornea with a matching second part of each crosscut incision in the donor tissue.

13. The method of claim 12, wherein the crosscut incisions are made at an anterior corneal surface of each cornea.

14. The method of claim 12, wherein the crosscut incisions made in each cornea do not extend through either cornea.

15. The method of claim 12, wherein the crosscut incisions made in each cornea are equally spaced about the sidecut incision.

16. The method of claim 12 further comprising placing a plurality of sutures, each suture being placed along the aligned parts of one of the crosscut incisions.

17. The method of claim 12, wherein the incisions are made using a surgical laser.

18. A system for resecting corneal tissue, the system comprising:
a surgical laser adapted to emit a pulsed laser beam;
a focusing assembly adapted to focus the pulsed laser beam into a cornea;
an interface adapted to provide a plurality of incision patterns for selection of a sidecut pattern and provide options for selection of a crosscut incision quantity; and
a controller in communication with the interface to receive the sidecut pattern and the number of crosscut incisions from the interface, the controller being adapted to
move a focal point of the pulsed laser beam within the cornea using the focusing assembly,
direct the focal point of the pulsed laser beam to make a sidecut incision in the cornea according to the sidecut pattern, wherein the corneal tissue being resected is at least partially bounded by the sidecut incision, and
make a plurality of crosscut incisions according to the crosscut incision quantity, the crosscut incisions being incised across the sidecut incision.

19. The system of claim 18, wherein the crosscut incisions are equally spaced about the sidecut incision.

20. The system of claim 18, wherein the crosscut incisions do not extend through the cornea.

21. The system of claim 18, wherein the crosscut incisions are made at an anterior corneal surface.

22. A system for resecting corneal tissue, the system comprising:
a surgical laser adapted to emit a pulsed laser beam;
a focusing assembly adapted to focus the pulsed laser beam into a cornea;
an interface adapted to provide a plurality of incision patterns for selection of a resection pattern which includes a sidecut and a plurality of crosscuts which cross the sidecut; and
a controller in communication with the interface to receive the resection pattern, the controller being adapted to
move a focal point of the pulsed laser beam within the cornea using the focusing assembly, and
direct the focal point of the pulsed laser beam to make a resection incision in the cornea according to the resection pattern.

23. The system of claim 22, wherein the crosscuts are equally spaced about the sidecut.

24. The system of claim 22, wherein portions of the resection incision which correspond to the crosscuts do not extend through the cornea.

25. The system of claim 22, wherein of the resection incision which correspond to the crosscuts are made at an anterior corneal surface.
